(19) United States
(12) Patent Application Publication
(10) Pub. No.: US 2025/0115645 A1
(43) Pub. Date: Apr. 10, 2025

(54) AGGLOMERATED LEGUME PROTEINS AND METHODS FOR THEIR MANUFACTURE

(71) Applicant: CORN PRODUCTS DEVELOPMENT, INC., Westchester, IL (US)

(72) Inventors: Kamlesh SHAH, Bridgewater, NJ (US); Albert TILLETT, Bridgewater, NJ (US); Tushar SHAH, Bridgewater, NJ (US)

(21) Appl. No.: 18/291,325

(22) PCT Filed: Jul. 18, 2022
(86) PCT No.: PCT/US2022/037418

§ 371 Date: Jan. 23, 2024

Related U.S. Application Data
(60) Provisional application No. 63/226,097, filed on Jul. 27, 2021.

(51) Int. Cl. C07K 14/415; C07K 1/02

(52) U.S. Cl. CPC C07K 14/415; C07K 1/02

(57) ABSTRACT

The technology disclosed in this specification pertains to methods for agglomerating a legume protein isolate comprising heating a legume protein isolate to a temperature isolate to a temperature of at least about 35° C. applying an aqueous binding agent to the legume protein isolate to obtain the agglomerated legume protein isolate. In another aspect, the technology pertains to method for agglomerating a legume protein isolate comprising providing a first portion of a legume isolate; and applying a binding agent comprising a second portion of the legume protein isolate to the first portion of the legume protein isolate. Also disclose are agglomerated legume protein isolates obtained from the claimed methods.

The technology disclosed in this specification pertains to agglomerated legume protein isolates and methods for making them.

Legume protein isolates are a class of powdered food ingredients comprised substantially of isolated legume protein. Common processes for obtaining legume protein isolates begin with a legume-seed flour. The flour is dispersed in aqueous solution and the protein is separated from the other components of the flour by applying one or more of shear, high pH, and low pH to the dispersion. The separated protein product is then is recovered from solution and sold as a powder, having protein content that is commonly above 75% (wt. %). Notably, the extraction process denatures the legume protein, so legume protein isolates are substantially insoluble in water.

Being fine powders, it is common for legume protein isolate particles to clump loosely together making the product difficult to work with, particularly if the use relies in part on the uniform flow of the powdered product. Agglomeration is a process that intentionally binds one or more smaller particles together using a binding agent to form a larger particle size powder. Depending on the agglomeration process and the structure and composition of the agglomerates, an agglomerated powder may flow better. Common agglomeration processes use a binding agent comprised of water-soluble ingredients, dissolved in water or aqueous solution, to bind two or more particles in the base powder together.

As said, legume protein isolates are highly insoluble in water. The technology disclosed in this specifications, however, obtains agglomerated legume protein isolates using a binding agent comprised of water or a dispersion of agglomerated legume protein isolate in water. In embodiments where the binding agent uses a legume protein isolate legume protein isolate is a not modified by for example enzyme or acid or strong to adjust or improve the solubility of the protein binding agent such as by deamidation or by hydrolysis. The processes described in this specification effectively agglomerated base legume protein isolates to usefully alter the function of the base isolate, including by improving the flowability of the powder.

In any embodiment described in this specification, a method for agglomerating a legume protein isolate comprises, optionally, heating a legume protein isolate to a temperature of at least about 35° C. or to a temperature in a range of about 35° C. to about 125° C., or about 35° C. to about 120° C., or about 35° C. to about 115° C., or about 35° C. to about 110° C., or about 35° C. to about 105° C., or about 35° C. to about 100° C., or about 35° C. to about 95° C., or about 35° C. to about 90° C., or about 35° C. to about 85° C., or about 35° C. to about 80° C., or about 35° C. to about 75° C., or about 35° C. to about 70° C., or about 35° C. to about 65° C., or about 35° C. to about 60° C., or about 35° C. to about 55° C., or about 35° C. to about 50° C.; and applying an aqueous binding agent to the legume protein isolate to obtain the agglomerated legume protein isolate. In preferred embodiments the legume protein isolate is heated to a temperature or about 35° C. to about 55° C., or about 35° C. to about 50° C.

In any embodiment this specification describes a method for agglomerating a legume protein isolate comprises heating a legume protein isolate to a temperature of at least about 35° C. or to a temperature in a range of about 35° C. to about 125° C., or about 35° C. to about 120° C., or about 35° C. to about 115° C., or about 35° C. to about 110° C., or about 35° C. to about 105° C., or about 35° C. to about 100° C., or about 35° C. to about 95° C., or about 35° C. to about 90° C., or about 35° C. to about 85° C., or about 35° C. to about 80° C., or about 35° C. to about 75° C., or about 35° C. to about 70° C., or about 35° C. to about 65° C., or about 35° C. to about 60° C., or about 35° C. to about 55° C., or about 35° C. to about 50° C.; and applying an aqueous binding agent to the legume protein isolate to obtain the agglomerated legume protein isolate. In preferred embodiments the legume protein isolate is heated to a temperature or about 35° C. to about 55° C., or about 35° C. to about 50° C.

In some embodiments of the described method the binding agent comprises a portion of the same legume protein isolate that is to be agglomerated dispersed in water. In alternate embodiments of a method for agglomerating a legume protein described in this specification, the binding agent water.

In still another embodiment described in this specification, a method to be agglomerated a legume protein isolate described comprises providing a first portion of a legume isolate; and applying a binding agent comprising a second portion of the legume protein isolate to the first portion of the legume protein isolate to agglomerate the legume protein isolate.

With reference to embodiments using a binding agent comprising a legume protein in some embodiment the legume protein in the binding agent is a legume protein isolate. Although the legume protein isolate binding agent, may use any suitable legume protein isolate, in preferred embodiments, the legume protein isolate in the binding agent is the same legume protein isolate that is being agglomerated. In any embodiment of a method for agglomerating a legume protein isolate described in this specification, comprises obtaining a binding agent by mixing a legume protein isolate, preferably a portion of the same legume protein isolate to be agglomerated, and water.

Methods for making the legume protein isolate containing binding agent are now described. In any embodiment described in this specification, a legume protein isolate containing binding agent, comprises a legume protein isolate an amount of about 5% to about 20% (wt. % of the binding solution), or about 5% to about 15%, or about 6% to about 15%, or about 7% to about 15%, or about 8% to about 15%, or about 9% to about 15%, or about 10% to about 15%.

In embodiments described in this specification, a method for agglomerating a legume protein isolate comprises obtaining a binding agent by mixing an unmodified legume protein isolate in an amount of in an amount of about 5% to about 15% (wt. % of the binding solution), or about 6% to about 15%, or about 7% to about 15%, or about 8% to about 15%, or about 9% to about 15%, or about 9% to about 14%, or 9% to about 13%, or about 9% and about 12%, or about 9% to about 11%; and an aqueous solution prior to applying the binding agent to the legume protein isolate to agglomerate the legume protein.

In another embodiment of a method for agglomerating a legume protein isolate described in this specification a first portion of a legume protein isolate is agglomerated using a binding agent obtained by mixing a second portion of the legume protein isolate in an amount of about 5% to about 15% (wt. % of the binding solution), or about 6% to about 15%, or about 7% to about 15%, or about 8% to about 15%, or about 9% to about 15%, or about 9% to about 14%, or 9% to about 13%, or about 9% and about 12%, or about 9% to about 11%; and water prior to applying binding agent to the legume protein isolate.

In another embodiment of a method for agglomerating a legume protein isolate described in this specification a first portion of a legume protein isolate is agglomerated using a binding agent obtained by a process consisting essentially of mixing a second portion of the legume protein isolate in an amount of about 5% to about 15% (wt. % of the binding solution), or about 6% to about 15%, or about 7% to about 15%, or about 8% to about 15%, or about 9% to about 15%, or about 9% to about 14%, or 9% to about 13%, or about 9% and about 12%, or about 9% to about 11%; and water.

Legume protein isolates are not very soluble in water and do not stable disperse in water such that the legume protein isolate will fall out of solution quickly if not continuously stirred. So in at least some embodiments a binding agent is continuously mixed while it is actively being pumped into the agglomerating reactor to ensure a relatively constant amount of legume protein isolate is in the binding agent.

In any embodiments described in this specification a binding agent is or comprises water or aqueous solution having pH from about 7 to about 8. Whether using water as a binding agent or using binding agent comprising a legume protein isolate, and water or an aqueous solution, the binding agent has a pH from about 7 to about 8. Additionally, an agglomerated legume protein isolate obtained from the methods described in this specification has a pH from about 7 to about 8.

In various embodiments a method for agglomerating a legume protein isolate further comprises drying the agglomerated legume protein isolate to a moisture content from about 5% to about 10% (wt. % of the agglomerated legume protein isolate), or from about 6% to about 10%, or from about 6% to about 9, or from about 5% to about 9%.

Various reactors types (called in this specification an agglomerating reactor) are useful for practicing the methods for agglomerating legume protein isolates. In some embodiments, legume protein isolate is agglomerated according to the methods described in this specification using a hollow tube reactor in a process like that described in United States Publication number 2018/0192683 A1 at paragraph (United States Publication number 2018/0192683 A1 is incorporated by reference in its entirety into this specification). In other embodiments, a legume protein isolate is agglomerated using according to the methods described in this specification using fluid bed reactor or other similar device capable of fluidizing a batch of particulate matter using a fluidizing gas. Preferably the gas air, although inert gases may be used as the purpose is not to chemically react the particulate matter with the gas. During use, binding agent is sprayed into the reactor in atomized form and the mixture of binding agent and fluidized legume protein isolate interact forming agglomerated particles. In any embodiment described in this specification a method of agglomerating a legume protein comprising applying a binding agent to a legume protein isolate for a time from about 60 and 110 minutes or for a time in a range selected from the group consisting of i) about 90 and about 110 minutes; and ii) about 60 to about 80 minutes or about 65 to about 80 minutes, or about 65 to about 75 minutes; about 70 to about 75 minutes.

In another aspect, the technology disclosed in this specification pertains to an agglomerated legume protein isolate having defined size or flowability index. In any embodiment, this specification describes an agglomerated legume protein isolate having a particle size distribution having a D50 having a range selected from the group consisting of: i) from about 175 to about 225 microns, or from about 190 to about 210 microns; ii) from about 75 to about 125 microns, or from about 90 to 125 microns, or from about 100 to about 125 microns, and from about 100 to about 110 microns. Within this specification, particle size can be measured using a particle size analyzer, to assess percent of particles having a defined diameter. The D50 measurement, reports fiftieth percentile of diameters, meaning that 50% of diameters in the sample fall below a defined D50.

In any embodiment, this specification discloses an agglomerated legume protein isolate having a particle size distribution wherein the percent of agglomerated legume protein isolate particles having particle size greater than 100 microns is in a range selected from the group consisting of: i) about 50% to about 70% or about 60% to about 70%, or about 65% to about 70%; ii) about 50% to about 70% or about 50% to about 65% or about 50 to about 60% or about 50% to about 55%; and iii) at least about 90% or at least about 95% or at least about 96% or at least about 97%.

In any embodiment, this specification discloses an agglomerated legume protein isolate having a flowability index of greater than 65 or greater than 70.

In at least some embodiments an agglomerated legume protein isolate, as described in this specification has two or more attributes selected from the group consisting of: i) a particle size distribution having a D50 from about 175 to about 225 microns, or from about 190 to about 210 microns; ii) a particle size distribution having a percentage of particles greater than 100 microns of about 50% to about 70% or about 60% to about 70%, or about 65% to about 70%; and iii) a flowability index of greater than about 70.

In at least some embodiments an agglomerated legume protein isolate, as described in this specification has two or more attributes selected from the group consisting of: i) a particle size distribution having a D50 from about 175 to about 225 microns, or from about 190 to about 210 microns; ii) a particle size distribution having a percentage of particles greater than 100 microns about 50% to about 70% or about 50% to about 65% or about 50 to about 60% or about 50% to about 55%; and iii) a flowability index of greater than about 65.

In at least some embodiments an agglomerated legume protein isolate, as described in this specification has i) a particle size distribution of from about 75 to about 125 microns, or from about 90 to 125 microns, or from about 100 to about 125 microns, and from about 100 to about 110 microns; and ii) a particle size distribution having a percentage of particles greater than 100 microns of about at least about 90% or at least about 95% or at least about 96% or at least about 97%.

Legume protein isolates can be made from any legume sources. Preferred legumes include but are not limited to pea, fava bean, chickpea, lentil, and lupin. In at least some embodiments, an agglomerated legume protein isolate is an agglomerated pea protein isolate is made using the pea protein isolate as a protein binding agent. In any embodiment described in this specification, legume protein isolates are a dried powdered ingredient having a protein content greater than about 75% (wt. %) of the composition or from about 75% to about 95%, or from about 80% to about 85%. The foregoing are protein ranges apply to both the base legume protein isolate to be agglomerated and the agglomerated legume protein isolate.

The methods described in this specification are useful to obtain an agglomerated legume protein isolate having any attribute as described in this specification. Additionally this specification describes a legume protein isolate made by any process described in this specification and further comprising any attribute described in this specification.

Legume protein isolates are commercially available, but a generalized method use for obtaining a legume protein isolate that is a useful base material to make an agglomerated legume protein isolate is now described. In at least some embodiments described in this specification a legume protein isolate, preferably a pea protein isolate, is obtained using a process comprising adjusting the pH of a slurry comprising legume protein to alter the protein's solubility in water and relative to other components in the slurry so that the components can be separated.

In more detail, the starting legume protein isolate to be used in an agglomeration process can be obtained using what this specification calls “isoelectric point separation”. An illustrative method for obtaining legume protein isolate using the protein's isoelectric point comprises the steps laid out in this paragraph (but may include other steps). Legumes are milled to obtain a milled composition, for example a flour. The legumes are milled in a suitable process, including wet or dry milling processes. Following milling, the flour is dispersed in water to form an aqueous dispersion of flour. The aqueous dispersion is pH adjusted to alter the solubility of protein relative to other components of the flour. For example, at alkaline pH between 8 and 10 legume proteins are commonly very soluble in water compared to starch and fiber. The dissolved protein is separated from insoluble components using centrifugation or filters or similar process where the dissolved legume protein will be in a supernatant or effluent. The supernatant or effluent is pH adjusted to a pH where the legume protein is highly insoluble, for example at the isoelectric point of the legume protein. The isoelectric point differs for different protein sources. For at least some proteins, the isoelectric point or point where the proteins are least soluble in water is at pH between 4 and 5. The precipitate is recovered via centrifuge or filtering or similar process and is a legume protein isolate although it may go through various other preparation steps such as washing, pH adjustment, pasteurization and spray drying before the final product is obtained. The alkaline and acid pH's are chosen to avoid protein hydrolysis.

Within this specification, “aerated bulk density” refers to the density of an aerated or loose sample of the powder. While not intending to be limited to specific process for measuring aeriated bulk density, a useful process for measuring aerated bulk density uses a device that dispenses a sample into an agitated receptacle of defined volume. Once the sample is dispensed material overflowing the receptacle is removed so that the volume of the sample is equal to the volume of the receptacle. The receptacle is then weighed.

Within this specification, “packed bulk density” refers to the density of a powder in a receptacle of defined volume that has been agitated after the powder was dispensed to cause the powder to pack more closely in the receptacle. While not intending to be limited to specific process for measuring packed bulk density, a useful method for measuring packed bulked density uses a device that deposits a sample into a sleeved receptacle so that the sample overflows the receptacle. The receptacle is then agitated, such as by tapping the receptacle for a defined number of times. Once agitation is complete the sleeve is removed and then overflow material is removed so that the volume of material in the receptacle is equal to the volume of the receptacle. The receptacle is then weighed.

Within this specification, “compressibility index” is the percent difference between the aerated bulk density and the packed bulk density. It is calculated using the formula Compressibility index=(packed bulk density−aerated bulk density)/packed bulk density*100.

Within this specification, “flowability index” is a unitless value known in the art and measurable using Hosokawa Micron Powder Tester. The index is scaled from 0 to 100 with a higher number meaning better flowability, where flowability refers generally to the flow characters of a powder.

Use of “about” to modify a number is meant to include the number recited plus or minus 10%. Where legally permissible recitation of a value in a claim means about the value. Use of about in a claim or in the specification is not intended to limit the full scope of covered equivalents.

Recitation of the indefinite article “a” or the definite article “the” is meant to mean one or more unless the context clearly dictates otherwise.

While certain embodiments have been illustrated and described, a person with ordinary skill in the art, after reading the foregoing specification, can effect changes, substitutions of equivalents and other types of alterations to the methods, and of the present technology. Each aspect and embodiment described above can also have included or incorporated therewith such variations or aspects as disclosed regarding any or all the other aspects and embodiments.

The present technology is also not to be limited in terms of the aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to methods, conjugates, reagents, compounds, compositions, labeled compounds or biological systems, which can, of course, vary. All methods described herein can be performed in any suitable order unless otherwise indicated herein or otherwise clearly contradicted by context. It is also to be understood that the terminology used herein is for the purpose of describing aspects only and is not intended to be limiting. Thus, it is intended that the specification be considered as exemplary only with the breadth, scope and spirit of the present technology indicated only by the appended claims, definitions therein and any equivalents thereof. No language in the specification should be construed as indicating any non-claimed element as essential.

The embodiments illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the technology. This includes the generic description of the technology with a proviso or negative limitation removing any subject matter from the genus, regardless of whether the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member, and each separate value is incorporated into the specification as if it were individually recited herein.

The technology described in this specification can be further understood with reference to the following non-limiting examples that are provided for illustrative purposes and are not intended to limit the full scope of the invention.

1 A method for agglomerating a legume protein isolate comprising: optionally heating a legume protein isolate to a temperature isolate to a temperature of at least about 35° C. or to a temperature in a range of about 35° C. to about 125° C., or about 35° C. to about 120° C., or about 35° C. to about 115° C., or about 35° C. to about 110° C., or about 35° C. to about 105° C., or about 35° C. to about 100° C., or about 35° C. to about 95° C., or about 35° C. to about 90° C., or about 35° C. to about 85° C., or about 35° C. to about 80° C., or about 35° C. to about 75° C., or about 35° C. to about 70° C., or about 35° C. to about 65° C., or about 35° C. to about 60° C., or about 35° C. to about 55° C., or about 35° C. to about 50° C.; and applying an aqueous binding agent to the legume protein isolate to obtain the agglomerated legume protein isolate.

2 A method for agglomerating a legume protein isolate comprising: heating a legume protein isolate to a temperature isolate to a temperature of at least about 35° C. or to a temperature in a range of about 35° C. to about 125° C., or about 35° C. to about 120° C., or about 35° C. to about 115° C., or about 35° C. to about 110° C., or about 35° C. to about 105° C., or about 35° C. to about 100° C., or about 35° C. to about 95° C., or about 35° C. to about 90° C., or about 35° C. to about 85° C., or about 35° C. to about 80° C., or about 35° C. to about 75° C., or about 35° C. to about 70° C., or about 35° C. to about 65° C., or about 35° C. to about 60° C., or about 35° C. to about 55° C., or about 35° C. to about 50° C.; and applying an aqueous binding agent to the legume protein isolate to obtain the agglomerated legume protein isolate.

3 A method for agglomerating a legume protein isolate comprising: providing a first portion of a legume isolate; and applying a binding agent comprising a second portion of the legume protein isolate to the first portion of the legume protein isolate.

4 The method of any one of claims 1 to 3 wherein the legume protein isolate is heated to a temperature from about 35° C. to about 55° C. or from about 35° C. to about 50° C.

5 The method of any one of claims 1 to 4 wherein the legume protein isolate of step a) is a first portion of the legume protein isolate; and wherein the binding agent comprises a second portion of the legume protein isolate in an amount of about 5% to about 20% (wt. % of the binding solution), or about 5% to about 15%, or about 6% to about 15%, or about 7% to about 15%, or about 8% to about 15%, or about 9% to about 15%, or about 10% to about 15%.

6 The method of any one of claims 1 to 5 further comprising obtaining a binding agent by: mixing an unmodified legume protein isolate in an amount of in an amount of 5% to about 15% (wt. % of the binding solution), or about 6% to about 15%, or about 7% to about 15%, or about 8% to about 15%, or about 9% to about 15%, or about 9% to about 14%, or 9% to about 13%, or about 9% and about 12%, or about 9% to about 11%; and an aqueous solution prior to applying the binding agent according to step b).

7 The method of any one of claims 1 to 6 wherein the legume protein isolate of step a) is a first portion of the legume protein isolate and further comprising obtaining a binding agent by: mixing a second portion of the legume protein isolate in an amount of 5% to about 15% (wt. % of the binding solution), or about 6% to about 15%, or about 7% to about 15%, or about 8% to about 15%, or about 9% to about 15%, or about 9% to about 14%, or 9% to about 13%, or about 9% and about 12%, or about 9% to about 11%; and water prior to applying binding agent according to step b).

8 The method of any one of claims 1 to 7 wherein the binding agent consists essentially of: a second portion of the legume protein isolate in an amount of about 5% to about 15% (wt. % of the binding solution), or about 6% to about 15%, or about 7% to about 15%, or about 8% to about 15%, or about 9% to about 15%, or about 9% to about 14%, or 9% to about 13%, or about 9% and about 12%, or about 9% to about 11%; and water.

9 The method of any one of claims 1 to 8 wherein the binding agent is water.

10 The method of any one of claims 1 to 9 wherein the binding agent has a pH from about 7 to about 8.

11 The method of any one of claims 1 to 10 wherein the agglomerated legume protein isolate has a pH from about 7 to about 8.

12 The method of any one of claims 1 to 11 further comprising drying the agglomerated legume protein isolate to a moisture content from about 5% to about 10% (wt. % of the agglomerated legume protein isolate), or from about 6% to about 10%, or from about 6% to about 9, or from about 5% to about 9%.

13 The method of any one of claims 1 to 12 further comprising applying the binding agent in step b) for a time from about 60 and 110 minutes or for a time in a range selected from the group consisting of: i) about 90 and about 110 minutes; and ii) about 60 to about 80 minutes or about 65 to about 80 minutes, or about 65 to about 75 minutes; about 70 to about 75 minutes.

14 The method for any foregoing claim wherein the agglomerated legume protein isolate has an attributed as described in any one of claims 19 to 27.

15 The method of any one of claims 1 to 14 wherein the legume protein isolate that is to be agglomerated and, optionally, any legume protein isolate in the binding agent is selected from the group consisting of pea protein, chickpea protein, lentil protein, fava bean protein, and mixtures thereof.

16 The method of one of claims 1 to 15 wherein the legume protein isolate that is to be heated in step a) is a first portion of a pea protein isolate and the binding agent comprises a second portion of the pea protein isolate.

17 The method of any one of claims 1 to 16 wherein the legume protein isolate is agglomerated in a fluidizing bed reactor.

18 The method of any one of claims 1 to 17 wherein the binding agent is a mixture of legume protein isolate and water and the mixture is constantly mixed while the binding agent is pumped into the agglomerating reactor.

19 An agglomerated legume protein isolate obtainable by or obtained by a process as described in foregoing claim.

20 The agglomerated legume protein isolate as described in claims 19 further comprising any attribute as described in any one of claims 21 to 27.

21 An agglomerated legume protein isolate having a particle size distribution having a D50 having a range selected from the group consisting of: i) from about 175 to about 225 microns, or from about 190 to about 210 microns; and ii) from about 75 to about 125 microns, or from about 90 to 125 microns, or from about 100 to about 125 microns, and from about 100 to about 110 microns.

22 The agglomerated legume protein isolate of claim 21 having a particle size distribution wherein the percent of agglomerated legume protein isolate particles having particle size greater than 100 microns is in a range selected from the group consisting of: i) about 50% to about 70% or about 60% to about 70%, or about 65% to about 70%; ii) about 50% to about 70% or about 50% to about 65% or about 50 to about 60% or about 50% to about 55%; and iii) at least about 90% or at least about 95% or at least about 96% or at least about 97% wherein, optionally, the particle size is a sieved particle size.

23 The agglomerated legume protein isolate of claim 21 or 22 having a flowability index of greater than 65 or greater than 70.

24 The agglomerated protein isolate of any one of claims 21 to 23 having two or more attributes selected from the group consisting of: i) a particle size distribution having a D50 from about 175 to about 225 microns, or from about 190 to about 210 microns; ii) a particle size distribution having a percentage of particles greater than 100 microns of about 50% to about 70% or about 60% to about 70%, or about 65% to about 70%, wherein, optionally, the particle size is a sieved particle size; iii) a flowability index of greater than about 70.

25 The agglomerated protein isolate of any one of claims 21 to 24 having two or more attributes selected from the group consisting of: i) a particle size distribution having a D50 from about 175 to about 225 microns, or from about 190 to about 210 microns; ii) a particle size distribution having a percentage of particles greater than 100 microns about 50% to about 70% or about 50% to about 65% or about 50 to about 60% or about 50% to about 55%, wherein, optionally, the particle size is a sieved particle size; iii) a flowability index of greater than about 65.

26 The agglomerated protein isolate of any one of claims 21 to 25 having: i) a particle size distribution of from about 75 to about 125 microns, or from about 90 to 125 microns, or from about 100 to about 125 microns, and from about 100 to about 110 microns; and ii) a particle size distribution having a percentage of particles greater than 100 microns of about at least about 90% or at least about 95% or at least about 96% or at least about 97%, wherein, optionally, the particle size is a sieved particle size.

27 The agglomerated protein isolate of any one of claims 21 to 26 wherein the protein isolate is selected from the group consisting of pea, chickpea, fava bean, and lentil, wherein, preferably the agglomerated protein isolate is an agglomerated pea protein isolate.

28 A method for agglomerating a legume protein isolate comprising: heating a first portion of legume protein isolate to a temperature isolate to a temperature of at least about 35° C. or to a temperature in a range of about 35° C. to about 125° C., or about 35° C. to about 120° C., or about 35° C. to about 115° C., or about 35° C. to about 110° C., or about 35° C. to about 105° C., or about 35° C. to about 100° C., or about 35° C. to about 95° C., or about 35° C. to about 90° C., or about 35° C. to about 85° C., or about 35° C. to about 80° C., or about 35° C. to about 75° C., or about 35° C. to about 70° C., or about 35° C. to about 65° C., or about 35° C. to about 60° C., or about 35° C. to about 55° C., or about 35° C. to about 50° C.; and applying an aqueous binding agent to the legume protein isolate to obtain the agglomerated legume protein isolate wherein, optionally, the binding agent comprises a second portion of the legume protein isolate.

29 The method of claim 28 wherein the first portion of legume protein isolate is heated to a temperature from about 35° C. to about 55° C. or from about 35° C. to about 50° C.

30 The method of claim 28 or 29 wherein the binding agent comprises the second portion of the legume protein isolate in an amount of about 5% to about 20% (wt. % of the binding solution), or about 5% to about 15%, or about 6% to about 15%, or about 7% to about 15%, or about 8% to about 15%, or about 9% to about 15%, or about 10% to about 15%.

31 The method of any one of claims 29 to 30 further comprising obtaining the binding agent by: mixing (i) a second portion of the unmodified legume protein isolate in an amount of in an amount of about 5% to about 20% (wt. % of the binding solution) 5% to about 15%, or about 6% to about 15%, or about 7% to about 15%, or about 8% to about 15%, or about 9% to about 15%, or about 9% to about 14%, or 9% to about 13%, or about 9% and about 12%, or about 9% to about 11%; and (ii) an aqueous solution prior to applying the binding agent according to step b).

32 The method of any one of claims 29 to 31 wherein the binding agent consists essentially of: the second portion of the legume protein isolate in an amount of about 5% to about 15% (wt. % of the binding solution), or about 6% to about 15%, or about 7% to about 15%, or about 8% to about 15%, or about 9% to about 15%, or about 9% to about 14%, or 9% to about 13%, or about 9% and about 12%, or about 9% to about 11%; and water.

33 The method of any one of claims 29 to 32 wherein the binding agent is water.

34 The method of any one of claims 29 to 33 wherein the binding agent has a pH from about 7 to about 8.

35 The method of any one of claims 29 to 34 wherein the agglomerated legume protein isolate has a pH from about 7 to about 8.

36 The method of any one of claims 29 to 35 further comprising drying the agglomerated legume protein isolate to a moisture content from about 5% to about 10% (wt. % of the agglomerated legume protein isolate), or from about 6% to about 10%, or from about 6% to about 9, or from about 5% to about 9%.

37 The method of any one of claims 29 to 36 further comprising applying the binding agent in step b) for a time from about 60 and 110 minutes or for a time in a range selected from the group consisting of: i) about 90 and about 110 minutes; and ii) about 60 to about 80 minutes or about 65 to about 80 minutes, or about 65 to about 75 minutes; about 70 to about 75 minutes.

38 The method of any foregoing claim wherein the agglomerated legume protein isolate obtained from the method has an attributed as described in any one of claims 43 to 48.

39 The method of any one of claims 29 to 38 wherein the first and second portions of the legume protein isolate are selected from the group consisting of pea protein, chickpea protein, lentil protein, fava bean protein, and mixtures thereof.

40 The method of one of claims 29 to 39 wherein the first and second portions of the legume protein isolate are a pea protein isolate.

41 The method of any one of claims 29 to 40 wherein the legume protein isolate is agglomerated in a fluidizing bed reactor.

42 The method of any one of claims 29 to 41 wherein the binding agent is a mixture of legume protein isolate and water and the mixture is constantly mixed while the binding agent is pumped into an agglomerating reactor.

43 An agglomerated legume protein isolate having a particle size distribution having a D50 in a range selected from the group consisting of: i) from about 175 to about 225 microns, or from about 190 to about 210 microns; and ii) from about 75 to about 125 microns, or from about 90 to 125 microns, or from about 100 to about 125 microns, and from about 100 to about 110 microns.

44 The agglomerated legume protein isolate of claim 43 having a particle size distribution wherein the percent of agglomerated legume protein isolate particles having particle size greater than 100 microns is in a range selected from the group consisting of: i) about 50% to about 70% or about 60% to about 70%, or about 65% to about 70%; ii) about 50% to about 70% or about 50% to about 65% or about 50 to about 60% or about 50% to about 55%; and iii) at least about 90% or at least about 95% or at least about 96% or at least about 97% wherein, optionally, the particle size is a sieved particle size.

45 The agglomerated legume protein isolate of claim 43 or 44 having a flowability index of greater than 65 or greater than 70.

46 The agglomerated protein isolate of any one of claims 43 to 45 having two or more attributes selected from the group consisting of: i) a particle size distribution having a D50 from about 175 to about 225 microns, or from about 190 to about 210 microns; ii) a particle size distribution having a percentage of particles greater than 100 microns of about 50% to about 70% or about 60% to about 70%, or about 65% to about 70%, wherein, optionally, the particle size is a sieved particle size; iii) a flowability index of greater than about 70.

47 The agglomerated protein isolate of any one of claims 43 to 46 having two or more attributes selected from the group consisting of: i) a particle size distribution having a D50 from about 175 to about 225 microns, or from about 190 to about 210 microns; ii) a particle size distribution having a percentage of particles greater than 100 microns about 50% to about 70% or about 50% to about 65% or about 50 to about 60% or about 50% to about 55%, wherein, optionally, the particle size is a sieved particle size; iii) a flowability index of greater than about 65.

48 The agglomerated protein isolate of any one of claims 43 to 47 having: i) a particle size distribution having a D50 from about 75 to about 125 microns, or from about 90 to 125 microns, or from about 100 to about 125 microns, and from about 100 to about 110 microns; and ii) a particle size distribution having a percentage of particles greater than 100 microns of about at least about 90% or at least about 95% or at least about 96% or at least about 97%, wherein, optionally, the particle size is a sieved particle size.

49 The agglomerated protein isolate of any one of claims 43 to 48 wherein the agglomerated protein isolate is selected from the group consisting of pea, chickpea, fava bean, and lentil, wherein, preferably the agglomerated protein isolate is an agglomerated pea protein isolate.

50 The agglomerated pea protein of any of claims 43 to 49 made by a method as recited in any one of claims 29 to 42.

The technology described in this specification can be further understood with reference to the following non-limiting examples that are provided for illustrative purposes and are not intended to limit the full scope of the invention.

### EXAMPLE 1—AGGLOMERATED LEGUME PROTEIN ISOLATES (PROCESS VARIATIONS)

A total of six batches of agglomerated pea protein isolate were prepared using fluidizing bed reactors. Pea protein isolates used for agglomeration and as binding agent were spray dried material obtained using an isoelectric point separation. The agglomeration process was carried out in a Glatt 125 Fluid Bed Agglomerator Prior to agglomerated pea protein isolate reactor bowls were pre-heated at 100° C. for 15 minutes and surfaces swabbed with microbiological rapid test kits to ensure food contact surfaces were sterile. Upon completion, bowls were loaded with 220-lb (100-kg) of pea protein isolate (about 85% protein wt. %). Product was fluidized in reactor and heated to 40° C. Binder as then sprayed into the reactor to agglomerate the pea protein isolate at an air speed set to 1800-2000 cubic feet per minute (about 50 to about 57 cubic meters per minute) to achieve fluidization and after agglomeration the product as to a 6-8% (wt. %) moisture. Additional parameters for agglomerating are reported Table 1.

Binder solution was prepared in 25-gal (94.6 L) slurry tank. Binder was mixed with water until full dispersed at various solids concentration. Binder and pumped using a M2 double diaphragm pump. Specific binder mixtures evaluated are listed in Table 1.

TABLE 1
Processing Conditions for Aggolmerating Pea Protein Isoalte

| | Batch # | | | | | |
|---|---|---|---|---|---|---|
| | Batch 1 | Batch 2 | Batch 3 | Batch 4 | Batch 5 | Batch 6 |
| Pea Protein | 100 | 100 | 100 | 100 | 100 | 100 |
| Isolate (kg) | | | | | | |

BINDER

| | | | | | | |
|---|---|---|---|---|---|---|
| Pea Protein | 12 | 6.8 | 9.9 | 7.1 | 0 | 9.1 |
| Isolate (kg) | | | | | | |
| Wt. % Pea | 18% | 18% | 18% | 10% | 0% | 13% |
| Protein Isolate | | | | | | |
| in water | | | | | | |

PROCESS

| | | | | | | |
|---|---|---|---|---|---|---|
| Atomizing Air | 2.1 | 2.1 | 2.1 | 2.1 | 2.1 | 2.1 |
| Pressure (bar) | | | | | | |
| Intake Fan | 10 | 10 | 10 | 10 | 10 | 10 |
| (Hz) | | | | | | |
| Exhaust Fan | 30/35 | 35 | 35 | 35 | 35 | 37.5 |
| (Hz) | | | | | | |
| Inlet Air Temp | 108 | 121 | 122 | 122 | 92 | 123 |
| (° C.) | | | | | | |
| Exhaust Temp. | 45.6 | 40.8 | 40.5 | 47 | 48 | 50 |
| (° C.) | | | | | | |
| Binder Add | 95 | 41 | 66 | 72 | 63 | 101 |
| Time (min) | | | | | | |
| Drying Time | 10 | 5 | 10 | 10 | 10 | 10 |
| (min) | | | | | | |

### EXAMPLE 2—AGGLOMERATED LEGUME PROTEIN ISOLATES (PHYSICAL CHARACTERISTICS)

The six samples of agglomerated pea protein isolate made as described in Example 1 were evaluated for the following physical characteristics.

Particle size was measured using a particle size analyzer and sieved particle size distribution process. Table 2 reports particle sizes measured using a Malvern Mastersizer 3000 particle size analyzer, D10, D50 and D90. These respectively are the tenth, fiftieth and ninetieth percentiles for particle sizes. For example, with reference to D50, this represents particle size at which fifty percent of all particle sizes are small than the particle size reported.

Table 3 reports sieved particle size distribution results. The results were obtained by passing a batch of agglomerated pea protein isolate through sieves of successively smaller pore size. The amount retained on each sieve is measured for weight and is reported as a weight percentage retained on a sieve of defined size compared to the total weight of the sample. Results were obtained using a Tyler Ro-Tap Sieve Shaker equipped with timer and set of 8-inch sieves (about 20 cm). In operation the timer was set to five minutes and the sieves had mesh size of 20-mesh (850 μm), 40-mesh (425 μm), 100-mesh (150 μm), and 200 mesh (75 μm). Note that in Table 3, the among amount through 200 mesh is material that passed through the 200-mesh sieve. Total sample evaluated was 50 g.

Table 4 reports aerated bulk density, packed bulk density, compressibility index, dispersibility, and flowability index measured for the six samples of agglomerated legume protein made as describe in Example 1. These attributes were measured using a Hosokawa Micron Powder Tester.

Aerated bulk density refers to the density of an aerated or loose sample (g/ml) of the powder. The process used a device that dispenses a sample into an agitated receptacle of defined volume. In poor flowing powders, agitation as the sample is dispensed into the receptacle causes reduces how closely packed the particles are to each other in receptacle, reducing the number of particles that fit within the receptacle. Once the sample was completely dispensed, piled material overflowing the receptacle was removed so that material volume was equal to receptacle volume, and the receptacle's weight was measured.

Packed bulk density (sometimes called in the art tapped or tamped density) refers to the density (g/ml) of a powder in a receptacle of defined volume. The sample was deposited into a sleeved receptacle so that the sample overflowed the receptacle. The receptacle was then agitated, by tapping the receptacle for a defined number of times, causing the powder to redistribute in the receptacle in a more closely packed configuration. Once agitation was completed the sleeve was removed and then overflow material was removed so that the volume of material in the receptacle was equal to the volume of the receptacle. The receptacle was then weighed.

Compressibility index is the percent difference between the aerated bulk density and the packed bulk density. It was calculated using the formula Compressibility index=(packed bulk density−aerated bulk density)/packed bulk density*100. A large compressibility index means there is a large difference between the packed bulk density and then aerated bulk density indicating that the powdered material does not flow well into a closely packed configuration.

Flowability index is a unitless value known in the art and measurable using Hosokawa Micron Powder Tester. The index is scaled from 0 to 100 with a higher number meaning better flowability, where flowability refers general to the flow characters of a powder. In practice a powder tester derives flowability index by calculating four physical properties called compressibility (as described above) angle of repose (measures of the steepness of an angle relative to the horizontal that a pile of the powder can make on a flat fixed surface), angle of spatula (measures of the steepness of an angle relative to a spatula surface that a pile of the powder can make on the spatula) and cohesion (a measure of how much powder sticks to itself as it is passed through an agitated sieve). The four values are index individually according to a scale originally defined by R. L. Carr. The flowability index is the sum of the four indexed values.

Color measurements are reported in Table 5 and were obtained using a Hunter Color QUEST II spectrocolorimeter sphere model with Universal V.36 software and a NIR compression cell with quartz window.

TABLE 2
Agglomerated Product Laser Particle Size:
Malvern Mastersizer 3000 Instrument

| | Base | | | | | | |
|---|---|---|---|---|---|---|---|
| | (not | | | | | | |
| | agglom- | Batch # | | | | | |
| | erated) | Batch 1 | Batch 2 | Batch 3 | Batch 4 | Batch 5 | Batch 6 |
| D(10) | 11.939 | 32.649 | 21.084 | 19.368 | 27.418 | 27.888 | 17.531 |
| μm | | | | | | | |
| D(50) | 29.145 | 243.517 | 73.368 | 58.742 | 210.539 | 202.198 | 108.537 |
| μm | | | | | | | |
| D(90) | 64.172 | 668.656 | 654.174 | 598.464 | 727.050 | 932.687 | 773.879 |
| μm | | | | | | | |

TABLE 3
Agglomerated Pea Protein Isolate: Sieved Particle Size Distribution

| | Batch # | | | | | |
|---|---|---|---|---|---|---|
| | Batch 1 | Batch 2 | Batch 3 | Batch 4 | Batch 5 | Batch 6 |
| | (wt. %) | (wt. %) | (wt. %) | (wt. %) | (wt. %) | (wt. %) |
| on 20 mesh | 0.16 | 0.57 | 0.80 | 4.09 | 5.16 | 3.19 |
| (850 μm) | | | | | | |
| on 40 mesh | 25.40 | 15.11 | 16.23 | 24.36 | 23.96 | 20.66 |
| (425 μm) | | | | | | |
| on 100 mesh | 40.11 | 48.12 | 37.23 | 38.15 | 28.29 | 68.38 |
| (150 μm) | | | | | | |
| on 200 mesh | 13.35 | 27.60 | 35.75 | 15.96 | 19.66 | 5.47 |
| (75 μm) | | | | | | |
| thru 200 | 20.70 | 8.12 | 9.76 | 17.11 | 22.47 | 1.86 |
| mesh | | | | | | |
| (<75 μm) | | | | | | |

TABLE 4
Agglomerated Pea Protein Isolate Physical Characteristics

| | Base (not | Batch # | | | | | |
|---|---|---|---|---|---|---|---|
| | agglomerated) | Batch 1 | Batch 2 | Batch 3 | Batch 4 | Batch 5 | Batch 6 |
| Aerated Bulk | 0.0598 | 0.0892 | 0.0792 | 0.8333 | 0.0838 | 0.0884 | 0.0762 |
| Density (g/ml) | | | | | | | |
| Packed Bulk | 0.1145 | 0.1119 | 0.1233 | 0.1236 | 0.1091 | 0.1188 | 0.1150 |
| Density (g/ml) | | | | | | | |
| Compressibility | 49.5 | 20.3 | 35.8 | 32.6 | 23.2 | 25.6 | 33.7 |
| (%) | | | | | | | |
| Flowability | 48 | 67 | 60 | 51 | 72 | 67 | 56 |
| Index | | | | | | | |

Reviewing the data from Tables 2, 3, and 4 the following observations are made. Pea protein isolates obtained using an isoelectric point separation can be agglomerated using simple binding solutions with a process that heats the pea protein isolate before agglomeration. Also, pea protein isolates obtained using an isoelectric point separation can be agglomerated using a binding solution of the pea protein isolate and water wherein the pea protein isolate was not further modified to improve its solubility.

With reference to Batch 5 pea protein isolate can be agglomerated using water alone. Batch 5 has increased particle size and improved flowability compared to the base material. This process is useful for its simplicity but does not create the most flowable product.

With reference to Batch 4, obtaining the highest flowability index required using a binding solution of pea protein isolate and water. Notably best flowability is obtained when the binding solution uses pea protein isolate in an amounts less than about 15% (wt. %) dispersed in water. The without further modification.

With reference to Batch 6, larger sized agglomerated pea protein isolate can be obtained. Very large particle size may be useful in some applications; however the material has less flowability.

Table 5 provides the color distribution of agglomerated pea protein isolate using Hunter L, a, and b, colorimetry scale.

TABLE 5
Colorimetry Values of Agglomerated Pea Protein Isolate

| | Batch # | | | | | |
|---|---|---|---|---|---|---|
| | Batch 1 | Batch 2 | Batch 3 | Batch 4 | Batch 5 | Batch 6 |
| L-value | 78.81 | 81.79 | 82.19 | 79.37 | 79.67 | 82.08 |
| a-value | 3.39 | 2.48 | 2.34 | 3.21 | 3.09 | 2.45 |
| b-value | 20.89 | 19.31 | 19.00 | 20.48 | 20.44 | 18.06 |

The values are similar to, but generally darker than the base material.

1. A method for agglomerating a legume protein isolate comprising:
a. heating a first portion of legume protein isolate to a temperature in a range of about 35° C. to about 125° C.; and
b. applying an aqueous binding agent to the legume protein isolate to obtain the agglomerated legume protein isolate.

2. The method of claim 1 wherein the first portion of legume protein isolate is heated to a temperature from about 35° C. to about 55° C.

3. The method of claim 1 wherein the binding agent further comprises the second portion of the legume protein isolate in an amount of about 5% to about 20% (wt. % of the binding solution).

4. The method of claim 1 further comprising obtaining the binding agent by:
mixing (i) a second portion of the unmodified legume protein isolate in an amount of in an amount of about 5% to about 20% (wt. % of the binding solution); and (ii) an aqueous solution prior to applying the binding agent according to step b).

5. (canceled)

6. The method of claim 1 wherein the binding agent is water.

7. The method of claim 1 wherein the binding agent has a pH from about 7 to about 8.

8. (canceled)

9. The method of claim 1 further comprising drying the agglomerated legume protein isolate to a moisture content from about 5% to about 10% (wt. % of the agglomerated legume protein isolate).

10. The method of claim 1 further comprising applying the binding agent in step b) for a time from about 60 and 110 minutes.

11. (canceled)

12. The method of claim 1 wherein the legume protein isolate are selected from the group consisting of pea protein, chickpea protein, lentil protein, fava bean protein, and mixtures thereof.

13. The method of claim 1 wherein the legume protein isolate are a pea protein isolate.

14. The method of claim 1 wherein the legume protein isolate is agglomerated in a fluidizing bed reactor.

15. The method of claim 1 wherein the binding agent is a mixture of legume protein isolate and water and the mixture is constantly mixed while the binding agent is pumped into an agglomerating reactor.

16. An agglomerated legume protein isolate having a particle size distribution having a D50 in a range selected from the group consisting of:
i) from about 175 to about 225 microns
ii) from about 75 to about 125 microns.

17. The agglomerated legume protein isolate of claim 16 having a particle size distribution wherein the percent of agglomerated legume protein isolate particles having particle size greater than 100 microns is in a range selected from the group consisting of:
i) about 50% to about 70% or about 60% to about 70%, or about 65% to about 70%;
ii) about 50% to about 70% or about 50% to about 65% or about 50 to about 60% or about 50% to about 55%; and
iii) at least about 90% or at least about 95% or at least about 96% or at least about 97%

18. The agglomerated legume protein isolate of claim 16 having a flowability index of greater than 65.

19. The agglomerated protein isolate of claim 16 having two or more attributes selected from the group consisting of:
i) a particle size distribution having a D50 from about 175 to about 225 microns;
ii) a particle size distribution having a percentage of particles greater than 100 microns of about 50% to about 70%;
iii) a flowability index of greater than about 70.

20. The agglomerated protein isolate of claim 16 having two or more attributes selected from the group consisting of:
i) a particle size distribution having a D50 from about 175 to about 225 microns;
ii) a particle size distribution having a percentage of particles greater than 100 microns about 50% to about 70%;
iii) a flowability index of greater than about 65.

21. The agglomerated protein isolate of claim 16 having:
i) a particle size distribution having a D50 from about 75 to about 125 microns; and
ii) a particle size distribution having a percentage of particles greater than 100 microns of about at least about 90%.

22. The agglomerated protein isolate of claim 16 wherein the agglomerated protein isolate is selected from the group consisting of pea, chickpea, fava bean, and lentil.

23. (canceled)
